(19) United States
(12) Patent Application Publication
(10) Pub. No.: US 2009/0082790 A1
(43) Pub. Date: Mar. 26, 2009

(54) STERNAL CLOSURE DEVICE

(76) Inventors: Sujay Shad, New Delhi (IN); Lyn Gwyneth Lynette Scott, Bucks (GB); John William Wheeler, Hertfordshire (GB)

Correspondence Address: RENNER OTTO BOISSELLE & SKLAR, LLP, 1621 EUCLID AVENUE, NINETEENTH FLOOR, CLEVELAND, OH, 44115, US

(21) Appl. No.: 12/297,639

(22) PCT Filed: Apr. 17, 2007
(86) PCT No.: PCT/GB07/01391

§ 371 Date: Oct. 18, 2008

(30) Foreign Application Priority Data
Apr. 18, 2006 (GB) 0607608.7

(51) Int. Cl. A61B 17/04

(52) U.S. Cl. 606/148

(57) ABSTRACT

A device to facilitate post-operative sternal closure by twisting the opposite end regions of the suture wire together comprises spaced guides in the form of limbs (1 and 2) with passageways (3 and 4) through which the proximal wire ends (12) and (13) may be fed and a cross-piece (7) transversely positioned with respect to said guides whereby, in use, the wire end regions may be bent across and supported by the cross-piece. The cross-piece includes a discontinuity (8) with a spacing adapted to be less than the thickness of wire. As the tension in the wire increases during the course of the twisting motion a point will be reached when the wire end regions forcefully deform the cross-piece (7) enabling the wires to pass through the discontinuity. Thus an indication is given when a maximum tension is reached.

### FIELD OF THE INVENTION

This invention relates to a device that facilitates post-operative sternal closure using stainless steel wire suture.

### PRIOR ART

Sternal closure is a procedure for closing the sternum following mid-line sternotomy. Traditionally the sternum is cut down its centre and wired back together after the heart procedure by manual means using a stainless steel suture. One end of the suture firstly enters one side of the sternum wall (e.g. enters on the right-outside to inside) and exits through the opposing sternum wall (e.g. exits on the left-inside to outside). Force is applied to the suture to draw the two halves of the cut sternum together and manual twists are put in the wire to hold the sternum shut. Once closed, the twisted wire is cut and the cut end is folded down. It is usual for the wire to remain in place permanently.

Whilst this method is good, it is difficult to get a uniform twist which fully closes the sternum leaving no gaps. The invention provides a device which is aimed at simplifying that procedure, shortening closure time and providing a neater, cleaner closure which should lead to less patient discomfort. The device can be used in conjunction with existing needles and stainless steel sutures.

### SUMMARY OF THE INVENTION

According to this invention there is provided a device to facilitate post-operative sternal closure, using a wire suture, characterised in that the device comprises spaced guides through the respective ones of which the opposite end regions of the wire may be threaded, and a cross-piece so positioned with respect to said guides that, in use, the opposite regions of the wire emerging from the guides may be folded over the cross-piece.

Using such a device, and with the wire end regions threaded through to emerge from the guides past the cross-piece, the device can be moved downwards along the wire. This action shortens the distance between the device and the surface of the chest which draws the sternum walls within close proximity (analogy would be a drawstring on a child's coat). Final closure of the sternum wall occurs once the suture wire end regions are folded over and the device is twisted.

Preferably the cross-piece includes a means whereby the increase in the longitudinal pulling force exerted by the wire during twisting of the device can be sensed and/or indicated.

In a preferred embodiment also, the guides comprise spaced limbs through which the wire ends may be threaded.

The cross-piece may comprise a handle portion facilitating the twisting of the device to thus impart the twisting motion to the wire. It is not necessary for it to define a literally cross-shaped projection.

The cross-piece, in a most preferred embodiment, includes a discontinuity, or a weakened portion, adapted to be less than the thickness of wire. The cross-piece then is to be, to a greater or lesser extent, deformable. In this embodiment as the tension in the twisted length of wire increases during the course of the twisting motion a point will be reached when the wires forcefully deform the cross-piece and either increase the spacing of the discontinuity, or fracture the weakened portion, enabling the wires to pass therethrough. Thus both a visual and tactile indication is given when a maximum defined tension is reached.

Where the guides comprise limbs capable of flexure a lateral force across the limbs as the wires are twisted deform the limbs providing a failsafe mechanism.

In use, a device embodying the invention rotates the two ends of the wire one about the other in a twisting motion and a smooth action that draws the two sternum halves together. The torque is regulated to prevent the wires cutting through the bone. There is a reduction in back strain on the physician; a reduction in the time taken for closure, and a standardisation in the closure technique.

### BRIEF DESCRIPTION OF THE DRAWINGS

In the accompanying drawings:—

FIG. 1 shows a perspective view of a device in accordance with this invention,

FIG. 2 shows one half of the device illustrating the internal guides for the wire end regions,

FIGS. 3 through 6 show four consecutive stages in the use of the device, and

FIG. 7 shows another device embodying the invention.

### DESCRIPTION OF PREFERRED EMBODIMENTS

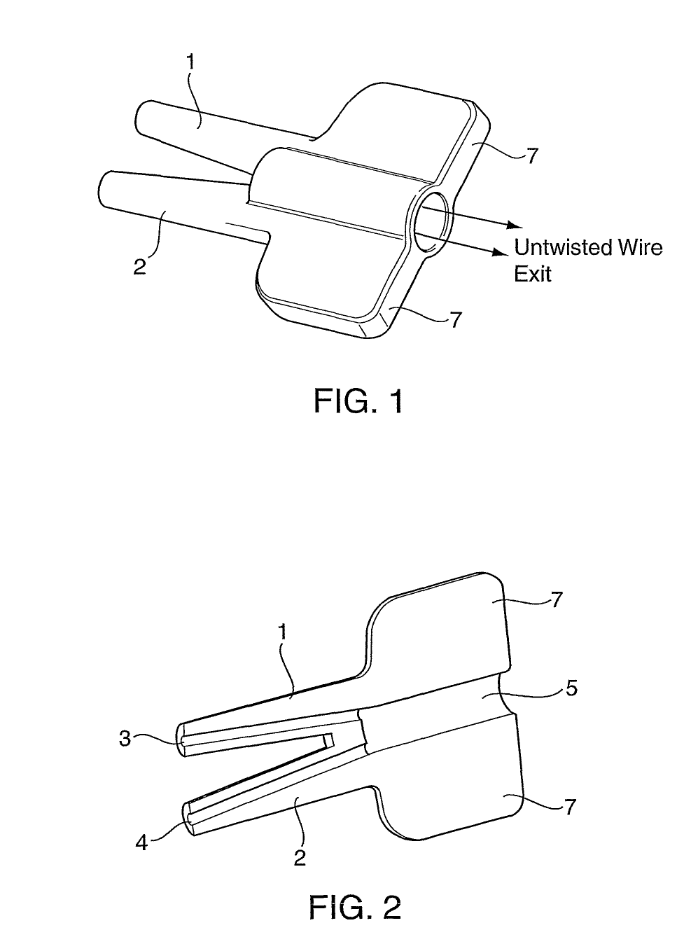

Referring to FIGS. 1 and 2, one device according to this invention comprises respective spaced tubular limbs 1 and 2 each of which has an internal guide channel 3 and 4 through which respective wire ends are threaded. These pass through a juncture zone 5 whilst remaining individually separate before emerging out the top surface of a handle part 6 which includes a cross-piece 7.

As seen in FIG. 2 the device is made from two identical moulded parts which are bonded together. The construction is of plastics material. In practice the device will more likely be a one-part moulding and will not necessarily be in two halves.

A surgeon using the device will puncture the patient's sternum bone in the usual way with a typical triangular reverse cutting needle (or other) attached to a suture, threading the wire through the sternum. The other side of the sternum wall is then punctured and the same wire threaded through. The suture needle is then cut off.

With the needle cut off, the wire suture ends can be threaded respectively into the spaced limbs of the device. This is either one end for a pre-threaded device or both ends for a separate device. In the preferred embodiment of the device, the wire ends go through some sprung plastic to keep a little bit of tension on them. The device can then be toggled down the wire to close the sternum.

At this point the two halves of the sternum are closed, but not necessarily tight together. If the sternum needs to be opened again, it can be, by sliding the device back up the wires.

Once closed, the ends of the wire can be carefully folded over the top surfaces of the cross-piece. When the device is then twisted, tension acts on the two ends of the wire to make a neat wire twist.

When the wire is further twisted, by twisting the body of the device, the patient's chest cavity is drawn closer together. The gap between the two limbs allows a final force to be applied which ‘nips’ the sternum closed. If too much force is applied, i.e. the sternum is fully closed, yet the wire wants to pull more, the limbs flex at a pre-determined load, to ensure a failsafe mechanism so that the bone is not cut by the suture.

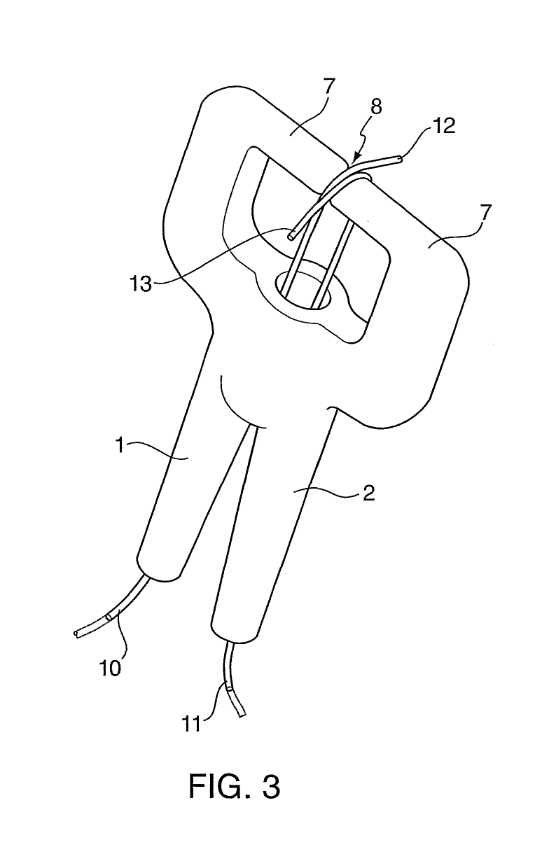

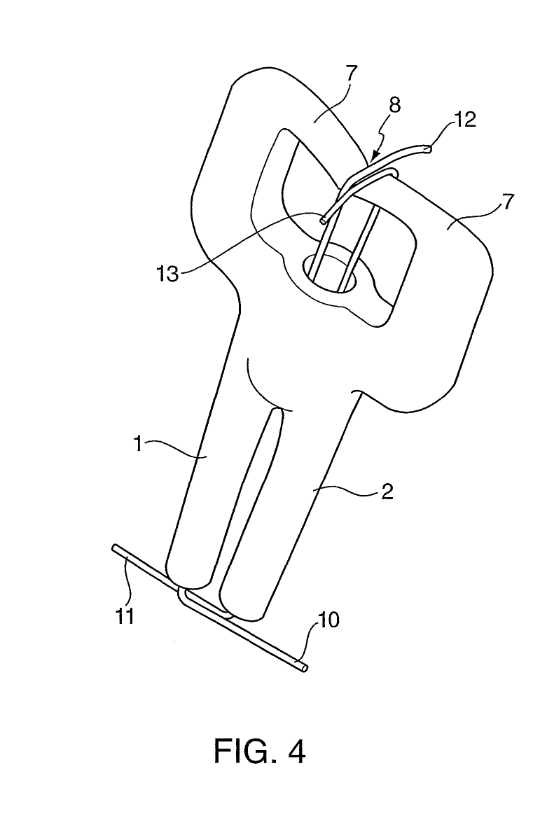

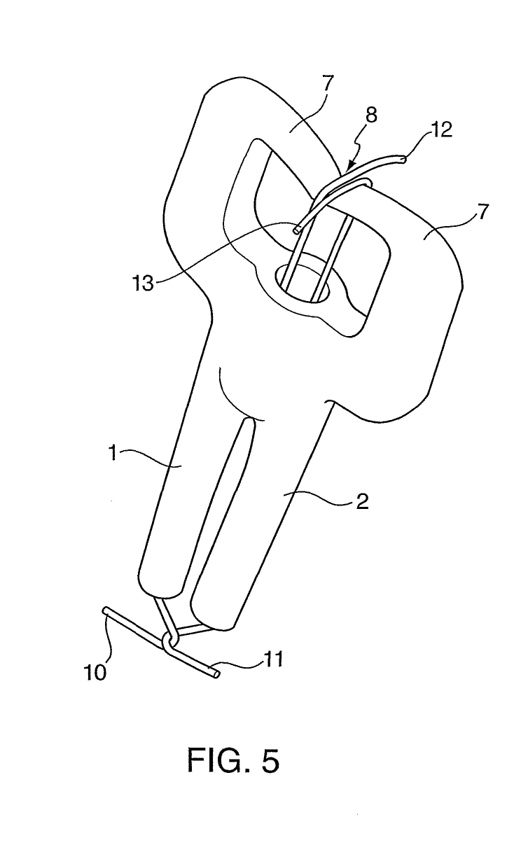

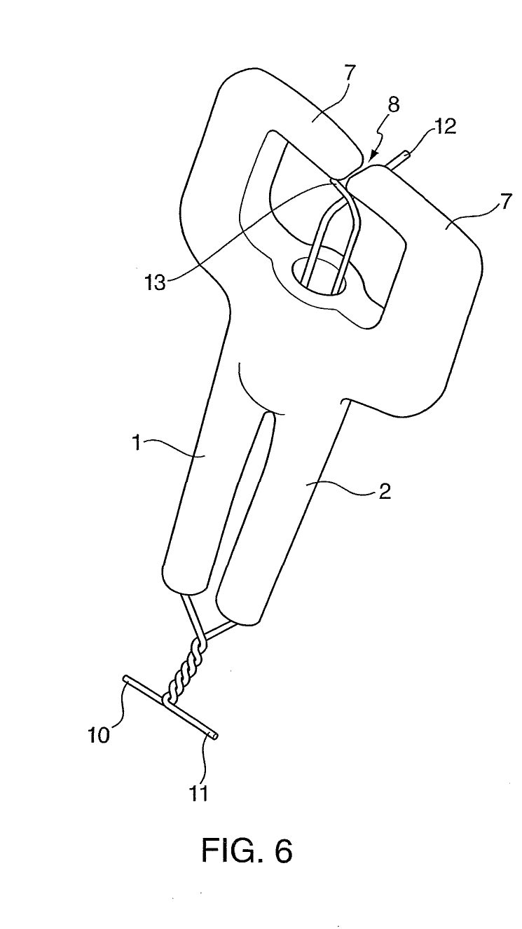

Referring now to FIGS. 3 through 6, the ends 12 and 13 of the wire are bent across the cross-piece 7 in the zone of a discontinuity 8 formed in the cross-piece. The limbs 1 and 2 may then come together as the device is initially turned whereby the wire end regions 10 and 11 adopt the position shown in FIG. 4. It will be noted here that the cross-piece is beginning to deform. FIG. 5 shows the first twist applied to the wire through rotation of the device with further deformation of the cross-piece. FIG. 6 shows the final position wherein the force on the proximal ends of the wire is sufficient for the cross-piece to have deformed to the point where the discontinuity 8 opens to allow the wires to pass. This may provide a visual and tactile indication that sufficient tension has been achieved.

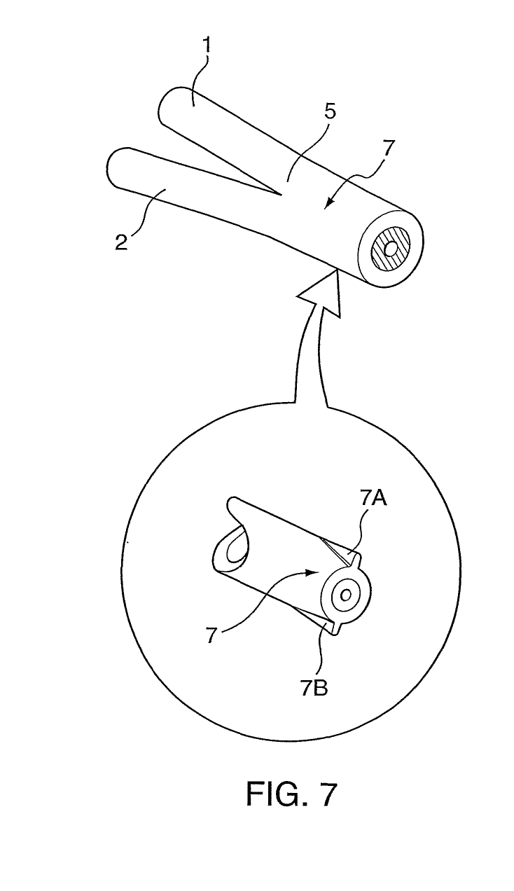

Finally FIG. 7 shows an alternative embodiment in which the so-called cross-piece 7 is in fact defined merely by an extension of the circular-cylindrical region 5 at which the guide-encasing limbs 1, 2 come together. A piece of spring plastic, indicated by diagonal shading in FIG. 7, occupies the end region of the circular-cylindrical handle 7 and is so dimensioned that the 2 wire-ends will pass through it; but when they are twisted together, above the handle 7, the twisted portion will not.

The external circular-cylindrical surface of the handle 7 is deliberately left unroughened because, in practice, it is envisaged that the surgeon's gloved fingers will be able to twist it adequately. By contrast in a sketch part-view that forms part of FIG. 7 as illustrated, fins 7A, 7B are cast into the handle moulding 7 to assist the twisting motion.

Several parameters are involved in the degree of tension finally achieved, including any or all of the width of the discontinuity in the cross-piece; the flexibility of the cross-piece; the characteristics of the wire, particularly the thickness; and the relative dimensions of the device. These parameters may all be ascertained empirically by those skilled in the art and adapted to specific needs.

One potential risk of the device is friction against the bone itself or bone damage by the action of the device on its surface. This can be eliminated by addition of a friction cup. The friction cup can be a simple tube with flat end through which the device sits with its legs inside. The top of the tube rests against the main body of the device. Thus, if any downward pressure needs to be applied by the surgeon, then the bone will not be damaged.

1. A device to facilitate post-operative sternal closure, using a wire suture, characterised in that the device comprises spaced guides through the respective ones of which the opposite end regions of the wire may be threaded, and a cross-piece so positioned with respect to said guides that, in use, the opposite end regions of the wire emerging from the guides may be folded over and supported by the cross-piece.

2. A device in accordance with claim 1, including a means whereby the desired longitudinal pulling force exerted by the wire during twisting of the device can be sensed and/or indicated.

3. A device in accordance with claim 1, wherein the cross-piece comprises a handle portion facilitating the twisting of the device to thus impart the twisting motion to the wire.

4. A device in accordance with claim 1, wherein the cross-piece includes a discontinuity with a spacing adapted to be less than the thickness of wire.

5. A device in accordance with claim 1, wherein the cross-piece includes a weakened portion which is capable of fracturing when the tension on the wire exceeds a defined value.

6. A device in accordance with claim 4, wherein the cross-piece is to a greater or lesser extent deformable and arranged such that as the tension in the twisted length of wire increases during the course of the twisting motion a point will be reached when the wires forcefully deform the cross-piece and either increase the spacing of the discontinuity or fracture the weakened portion, as appropriate.

7. A device in accordance with claim 1, wherein the spaced guides are capable of flexure such that a lateral force across the guides as the wires are twisted will deform the guides.

8. A surgical device as substantially described herein with reference to and as illustrated in the accompanying drawings.

9. A method of post-operative sternal closure substantially as described herein.
